(19) United States
(12) Patent Application Publication
(10) Pub. No.: US 2006/0236760 A1
(43) Pub. Date: Oct. 26, 2006

(54) Sports activity viewing, sensing, and interpreting system

(76) Inventor: William Redvers Belisle, New Orleans, LA (US)

Correspondence Address: WILLIAM REDVERS BELISLE, P.O. BOX 792653, NEW ORLEANS, LA, 70179, US

(21) Appl. No.: 11/114,435

(22) Filed: Apr. 26, 2005

(51) Int. Cl. A61B 5/22

(52) U.S. Cl. 73/379.04

(57) ABSTRACT

A sporting activity viewing, sensing, interpreting, seeing, reviewing, and replaying system comprised of an apparatus, system, and method for viewing, sensing, interpreting, seeing, reviewing, and replaying the sporting action occurring, occurred, or replayed regarding the sporting activity. The apparatus may be positioned below, within, or level to the sporting activity surface. The apparatus may incorporate or use optical, fiber, wire, radiation/radiological, physical, sound, motion, reflective, refractive, magnetic, electronic, smell, feel, computer, technological, digitized or non-digitized, transmitted, transferred, transported, pressure, temperature, mass, content, or other methods to view, sense, or interpret sport activities on grass, turf, soil, clay, silt, sand, concrete, wood, ice, asphalt, glass or plastic, rubber, ground, or components thereof, surfaces including artificial surfaces of these types. The degree to which sports activities are viewed, sensed, interpreted, seen, reviewed, or replayed may be enhanced considering present modes of the same.

### STATEMENT REGARDING FEDERALLY SPONSORED RESEARCH OR DEVELOPMENT

Not Applicable

### BACKGROUND OF THE INVENTION

1. Field of the Invention/Technical Field

The following is ‘A statement of the field of art to which the invention pertains:

1. U.S. Class: 33

Field of Search: 33/289

2. U.S. Class: 473

Field of Search: 473/490

3. U.S. Class: 367

Field of Search: 367/111

4. U.S. Class: 428

Field of Search: 428/17; 428/15; 428/86; 428/88; 428/92; 428/95; 428/137;

273/DIG.13

5. U.S. Class: 427

Field of Search: 427/7; 427/256

6. U.S. Class: 482

Field of Search: 482/54; 198/456

7. U.S. Class: 405

Field of Search: 405/37; 137/78.2; 137/561R; 405/36; 405/130; 405/131

8. U.S. Class: 405

Field of Search: U.S. Class: 405/37; 405/38; 405/43; 405/51

9. U.S. Class: 47

47/58.1R; 47/1.01F; 47/1.01R; 47/2; 405/37; 472/92

10. U.S. Class: 47

Field of Search: 47/48.5; 47/1.01F; 47/62R; 405/37

2. Description of the Related Art

Amron, et al. (2004) describe an apparatus, involving a first and second laser disposed at elevated locations and located on opposite lateral sides of the surface, for providing at least one temporary visible reference line on a non-smooth surface within the view of at least one video camera. Betts, et al. (2004) describe a method of fishing which comprises a stream of digital electric sync pulses used to find fish. Squires (2005) describes an artificial turf system that may be in multiple layers occurring on a base composed of ground elements with at least one impermeable layer for drainage and a filter that prevents particles from reaching the impermeable layer. Brogger, et al. (2001) describe a method of marking an article for retrospective authentication, by forming a unique mark including a set of microparticles, each said microparticle having two or more distinguishable layers with the sequence of the layers having an assigned meaning and applying the unique mark to an article to be authenticated, said mark being unique as a result of the pattern formed by the spatial relationship of said microparticles and said pattern being determined upon placement of said mark on the article. Mitchell (2000) describes a means for detecting motion of an object in any direction on the surface of a treadmill, comprising processing and imaging means arranged to provide the illusion of an environment of which the treadmill surface forms a part, said processing means using data concerning the motion of the object and outputting image data to the imaging means to create one or more images to present to the object.

Motz, et al. (1999) describe a control system for an athletic field that coordinates drainage by gravity or vacuum-enhanced drainage and irrigation by monitoring the water level with respect to a subsurface membrane, wherein a flow network resides on the membrane and is covered by a fill layer, which in turn supports the field surface thereabove. The flow network includes couplings located at the intersections of some of the pipe rows and conduit rows. The water permeability of the conduit rows allows the flow network to be used for draining, irrigating or heating the field. The heating of the fill layer and the surface thereabove minimizes energy costs and eliminates installation and maintenance costs that would otherwise be necessitated by separate heating and draining systems.

Motz, et al. (1998) describe a drainage system for a natural turf athletic field that includes a buried drainage network located above a water impermeable membrane which conforms to the topography of a compacted subsoil, the drainage network including a plurality of water impermeable pipe rows partially recessed in depressions in the subsoil and a plurality of water permeable conduit rows oriented perpendicular to and intersecting the pipe rows.

Cunningham (1984) describes a synthetic surface, such as artificial turf, rubberized asphalt, concrete composition, particulate mixtures and the like, that is applied on top of a subsurface base system, which in turn is on top of a subgrade. A liquid impervious membrane is positioned between the subgrade and the base system. The base system comprises an upper layer of sand-containing particulate material in which liquid characteristically moves in the vertical direction and a lower layer of gravel in which liquid characteristically moves well in the horizontal direction as well as downwardly. The material of the upper layer does not significantly penetrate into the gravel. The base system has a non-rutting upper surface. The non-rutting surface is accomplished by planting grass, cutting the grass at least once, and then killing the grass. The synthetic surface is then applied. A conduit system is positioned in the lower gravel layer of the base system, and a water reservoir is established in the base system. In warm temperatures, the synthetic surface can be cooled by maintaining the upper layer of the base system moist, and by circulating cool water into the reservoir and withdrawing warm water therefrom. Conversely, in cold temperatures, warm water can be circulated into the reservoir and colder water therein removed.

Cunninghgam (1981) describes a level unobstructed grass sports and recreation surface wherein the grass is deeply rooted in a layer of sand overlying a layer of gravel and preferably nurtured from below to provide a controlled growing environment exhibiting vigorous growth. The sand has more positive vertical liquid flow characteristics whereas the gravel has better horizontal liquid flow characteristics and the appropriate gradation selection of both determines the high drainage ability and uniform fertilizer/irrigation distribution throughout the system. The grass surface is defined by a water tight membrane extending from the perimeter, adjacent the surface and completely underlying the gravel layer. Control means are provided to detect the liquid level contained within the membrane and add or remove liquid to maintain the appropriate level. The buried conduit system is used for the controlled addition of water/fertilizer and excess drainage removal. The nutrified liquid from the reservoir is hydroponically supplied to the roots of the grass on a continuous demand basis. Heating cable may be placed at the boundary zone on top of the gravel and sand layers to warm the sand to remove frost and melt snow. An insulation layer may be placed beneath the membrane to minimize frost penetration to the subgrade. The system may be either completely or automatically programmed to accommodate external conditions such as degrees of use of the field, climatic conditions or the like or alternatively may be partially automatic and supported by more standard methods. Coupled with the control system can be a sensing unit to determine automatically the nature of the contained liquid.

The patents and information described above do not describe a method, apparatus, or system for sensing, viewing, or interpreting sporting activities from below, within, or at the surface on which the sporting activities occur.

To overcome these shortcomings, the present invention provides a method, apparatus, or system for sensing, viewing, or interpreting sporting activities from below, within, or at the surface on which the sporting activities occur.

### BRIEF SUMMARY OF THE INVENTION

It is the objective of the invention to provide an enhanced method of viewing, sensing, and interpreting sporting activities from below, within, and at the sporting activity surface.

### BRIEF DESCRIPTION OF SEVERAL VIEWS OF THE DRAWINGS

The present invention will be more fully understood by references to the following brief description thereof when read in conjunction with the attached drawings, and wherein:

FIG. 1. View of a section of a grass or turf sport activity surface showing a viewing, sensing, interpreting, seeing, reviewing, or replaying device in, on, or about a grass or turf “blade” positioned in or on the soil or other material and showing a surface on which the soil or other material may be positioned, the latter occurring in some indoor and some outdoor sports activity facilities or surfaces and includes the energy source if and as required. The viewing, sensing, interpreting, seeing, reviewing, or replaying device is shown positioned in, on, or about the soil or other material as well as in, on, or about the surface on which the soil or turf may be positioned. Energy sources are provided if and as required.

### DETAILED DESCRIPTION OF THE INVENTION

(References are to Illustrations)

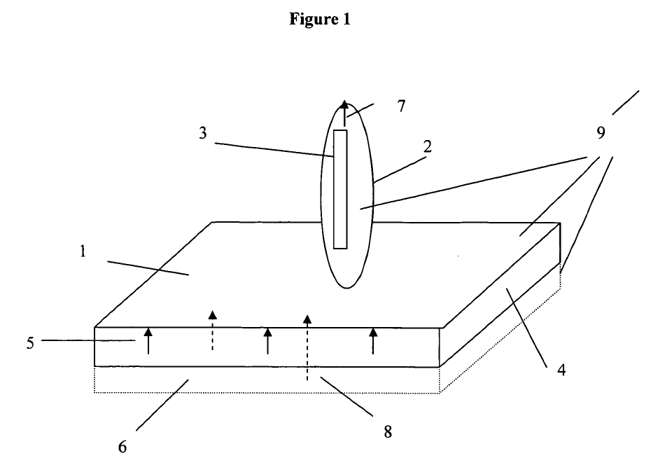

The sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system may be constructed beneath, within, or at the surface on which sporting activities occur. FIG. 1 shows a section of a surface on which a sporting activity may occur. Depicted in this figure are the surface of the material about which the grass or turf may be positioned (1), a grass or turf “blade” or other grass or turf material (2), the sensing, viewing, interpreting, seeing, reviewing, or replaying device positioned within a grass or turf “blade” as an example of device positioning, (3), a depth of the material in or about which the grass or turf blade may be positioned such as soil, ground components, or other material (4), the sensing, viewing, or interpreting device (5) positioned in or about the material on or about which the material in or on which the grass or turf “blade” or other grass or turf material may be positioned, the material on or about which the soil or other material (i.e. in or about which the grass or turf may be positioned) may be positioned (6), the sensing, viewing, interpreting, seeing, reviewing, or replaying device extending to and beyond the edges of the grass or turf “blade” or other grass or turf material (7), and the sensing, viewing, interpreting, seeing, reviewing, or replaying device positioned (8) in or about the base upon which the material in which the grass or turf blade is positioned. The energy source, within, about, or external to the grass or turf, as required, is also shown (9).

The surface in or about which the grass or turf may be positioned (1 and 4) may be grass, turf, soil, clay, silt, sand, concrete, rock, metal, wood, ice, asphalt, rubber, glass or plastic, ground, artificial, or components thereof sensing or various compositions of these materials. The surface in or about which the grass, turf, or other material may be positioned may be of a permanent or temporary nature.

The grass “blades” or other grass materials (2) are made of, and are representative of, one or more of the various kinds of grass on or about which sports activities may occur. The turf “blades” or other turf materials (2) are made of, and are representative of, one or more of the various materials of which turf sports surfaces are typically or non-typically made.

The sensing, viewing, interpreting, seeing, reviewing, or replaying device (3) positioned within or about a grass or turf “blade” or other grass or turf material, and the extension of the same (7), may be constructed, made, or grown in, on, or about the various materials that make up grass or turf “blades” or other grass or turf components upon or about which sporting activities occur. The sensing, viewing, interpreting, seeing, reviewing, or replaying device may be made or constructed using optical, fiber, wire, radiation/radiological, physical, sound, motion, reflective, refractive, magnetic, electronic, smell, feel, computer, technological, digitized or non-digitized, transmitted, transferred, transported, pressure, temperature, mass, or content sensing, viewing, or interpreting mechanisms. The sensing, viewing, interpreting, seeing, reviewing, or replaying devices or mechanisms may be positioned, constructed, or made within or about the grass or turf “blade” or other grass or turf material, as part of the grass or turf, or within or about the material in, on, or about which the grass or turf is positioned.

The surface or material in, on, or about which sporting activity grass or turf is positioned (1, 4) is usually soil, ground, or artificial material. The sensing, viewing, interpreting, seeing, reviewing, or replaying devices (5) may be made, constructed, or positioned in, on, or about said soil or other material in order to view the sporting activity from below the surface on which the sporting activity is occurring. The sporting activity sensing, viewing, interpreting, seeing, reviewing, or replaying device may view, sense, interpret, see, review, or replay the sporting activity from a single position or from multiple positions within or about the soil or other material as well as from a combination of positions within or about the grass, turf, soil, clay, silt, sand, concrete, rock, metal, wood, ice, asphalt, rubber, glass or plastic, ground, artificial, or components thereof or other material individually or in a combined manner. Similarly, the sensing, viewing, interpreting, seeing, reviewing, or replaying devices and mechanisms (8) may be positioned in, on, or about the material (6) upon which the materials in, on, or about which the grass or turf may be positioned (1, 4). Energy sources (9) may vary and are provided if needed and as required.

The sensing, viewing, interpreting, seeing, reviewing, or replaying device including the optical, fiber, wire, radiation/radiological, physical, sound, motion, reflective, refractive, magnetic, electronic, smell, feel, computer, technological, digitized or non-digitized, transmitted, transferred, transported, pressure, temperature, mass, or content sensing, viewing, or interpreting mechanisms may be positioned or may occur at any point in or about the grass or turf “blade” or other grass or turf material, the material in, on, or about which the grass or turf may be positioned, or at any point in or about the material on which the previously described material may be positioned.

### CROSS-REFERENCES TO RELATED APPLICATIONS:

1. Amron, Alan and Dinicola, Brian K., 2004, “System for operating one or more lasers to project a visible line onto a grass-covered surface”, U.S. Pat. No. 6,796,041.
2. Betts, David A., Bennett, Christopher James, Rasmussen, Douglas W., 2004, “Fish finding method and system”, U.S. Pat. No. 6,771,562.
3. Squires, Troy, 2005, “Horizontally draining, pre-engineered synthetic turf field”, U.S. Pat. No. 6,858,272.
4. Brogger, Brian and Kerns, William J., 2001, “System for retrospective identification and method of marking articles for retrospective identification”, U.S. Pat. No. 6,309,690.
5. Mitchell, Andrew John, 2000 “Motion apparatus”, U.S. Pat. No. 6,123,647.
6. Motz, Joseph E., Heinlein, Mark A., Goddard; James B., Tyner; Carl, Reese, Craig, Ferry, Brian L., 1999, “Control system for draining, irrigating and heating an athletic field”, U.S. Pat. No. 5,944,444.
7. Motz, Joseph E., Heinlein, Mark A., Goddard, James B., Tyner; Car, 1998, “Low profile drainage network for athletic field drainage system”, U.S. Pat. No. No. 5,752,784.
8. Cunningham, Percy C., 1984, “System for improving synthetic surfaces”, U.S. Pat. No. 4,462,184.
9. Cunningham, Percy C., 1981, “Grass sports surfaces and a method for maintaining them”, U.S. Pat. No. 4,268,993.

What I claim as my invention is:

1. A sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system comprising:
a device which allows the sensing, viewing, interpreting, seeing, reviewing, or replaying of athletic activities from below the sporting event surface,

2. The sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system, viewing, or interpreting system described in claim 1, wherein the sporting activities for which the system applies include golf, football, baseball, softball, basketball, tennis, soccer, rugby, table tennis or ping pong,

3. The sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system described in claim 1, wherein the system may sense, view, interpret, see, review, or replay a part of the sporting activity, parts of the sporting activity, or the entire sporting activity,

4. The sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system described in claim 1, wherein the system may sense, view, interpret, see, review, or replay the sporting activity on or above lines, edges, or markings on or about the sporting activity surface,

5. The sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system described in claim 1, wherein the method of sensing, viewing, interpreting, seeing, reviewing, or replaying involves optical, camera, fiber, wire, radiation/radiological, physical, sound, motion, reflective, refractive, magnetic, electronic, smell, feel, computer, technological, digitized or non-digitized, transmitted, transferred, transported, pressure, temperature, mass, or content methods.

6. The sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system described in claim 1, wherein the method of sensing, viewing, interpreting, seeing, reviewing, or replaying results may be of a composite or individual nature,

7. A sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system comprising:
a device which allows the sensing, viewing, interpreting, seeing, reviewing, or replaying of athletic activities from within the sporting event surface, including artificial surfaces of these types,

8. The sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system described in claim 7, wherein the sporting activities for which the system applies include golf, football, baseball, softball, basketball, tennis, soccer, rugby, table tennis or ping pong,

9. The sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system described in claim 7, wherein the system may sense, view, interpret, see, review, or replay a part of the sporting activity, parts of the sporting activity, or the entire sporting activity,

10. The sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system described in claim 7, wherein the system may sense, view, interpret, see, review, or replay the sporting activity on or above lines, edges, or markings on or about the sporting activity surface,

11. The sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system described in claim 7, wherein the method of sensing, viewing, interpreting seeing, reviewing, or replaying involves optical, camera, fiber, wire, radiation/radiological, physical, sound, motion, reflective, refractive, magnetic, electronic, smell, feel, computer, technological, digitized or non-digitized, transmitted, transferred, transported, pressure, temperature, mass, or content methods.

12. The sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system described in claim 7, wherein the method of sensing, viewing, interpreting, seeing, reviewing, or replaying results may be of a composite or individual nature,

13. A sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system comprising:
a device which allows the sensing, viewing, interpreting, seeing, reviewing, or replaying of athletic activities from a position level with the sporting event grass, turf, soil, clay, silt, sand, concrete, rock, metal, wood, ice, asphalt, rubber, glass or plastic, ground, artificial, or components thereof, surface,

14. The sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system described in claim 13, wherein the sporting activities for which the system applies include golf, football, baseball, softball, basketball, tennis, soccer, rugby, table tennis or ping pong,

15. The sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system described in claim 13, wherein the system may sense, view, interpret, see, review, or replay a part of the sporting activity, parts of the sporting activity, or the entire sporting activity,

16. The sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system described in claim 13, wherein the system may sense, view, interpret, see, review, or replay the sporting activity on or above lines, edges, or markings on or about the sporting activity surface,

17. The sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system described in claim 13, wherein the method of sensing, viewing, interpreting, seeing, reviewing, or replaying involves optical, camera, fiber, wire, radiation/radiological, physical, sound, motion, reflective, refractive, magnetic, electronic, smell, feel, computer, technological, digitized or non-digitized, transmitted, transferred, transported, pressure, temperature, mass, or content methods.

18. The sporting event grass, turf, soil, clay, silt, sand, concrete, rock, metal, wood, ice, asphalt, rubber, glass or plastic, ground, artificial, or components thereof, sensing, viewing, interpreting, seeing, reviewing, or replaying system described in claim 13, wherein the method of sensing, viewing, interpreting, seeing, reviewing, or replaying results may be of a composite or individual nature.
